(19) United States
(12) Patent Application Publication
(10) Pub. No.: US 2025/0140421 A1
(43) Pub. Date: May 1, 2025

(54) MORTALITY PREDICTION DEVICE AND METHOD FOR TRAUMA PATIENTS

(71) Applicant: University-Industry Cooperation Group of Kyung Hee University, Gyeonggi-do (KR)

(72) Inventors: JINSEOK LEE, Seoul (KR); SEUNG SEOK LEE, Gyeonggi-do (KR); HOON KO, Gyeonggi-do (KR); DONG KEON YON, Seoul (KR); SANG-HO LEE, Seoul (KR)

(21) Appl. No.: 18/933,257

(22) Filed: Oct. 31, 2024

(30) Foreign Application Priority Data
Nov. 1, 2023 (KR) 10-2023-0149348

(51) Int. Cl. G16H 50/30; G16H 50/70

(52) U.S. Cl. CPC G16H 50/30; G16H 50/70

(57) ABSTRACT

A mortality prediction device of trauma patients includes a data collection module that collects patient-related data of patients visiting an emergency department for a certain period of time, a learning data generation module that generates a learning data group for one patient by extracting a plurality of preset data from the patient-related data, and a prediction module that inputs the learning data group into one or more machine learning models, respectively, to learn the machine learning models to predict mortality of the corresponding patient.

### CROSS-REFERENCE TO RELATED APPLICATION AND CLAIM OF PRIORITY

This application claims the benefit under 35 USC § 119 of Korean Patent Application No. 10-2023-0149348, filed on Nov. 1, 2023, in the Korean Intellectual Property Office, the entire disclosure of which are incorporated herein by reference for all purposes.

### BACKGROUND

### 1. Technical Field

The examples of the present invention are related to mortality prediction technology of trauma patients.

### 2. Background Art

Trauma is one of the leading causes of death worldwide, and in particular, it is the leading cause under 45 years of age. Despite recent medical development, trauma-related mortality remains a significant problem. An Emergency Department (ED) of a hospital is the first point of contact with trauma patients, and decisions to reduce the golden time for treating patients with severe trauma should be made quickly and accurately. Therefore, a method to predict mortality for trauma patients vising an emergency department is required.

### SUMMARY

The examples of the present invention are to provide a new technique that can predict mortality of trauma patients.

The mortality prediction device of trauma patients according to one example disclosed, is a mortality prediction device of trauma patients equipped with one or more processors, and a memory storing one or more programs executed by the one or more processors, which comprises a data collection module that collects patient-related data of patients visiting an emergency department for a certain period of time; a learning data generation module that generates a learning data group for one patient by extracting a plurality of data preset from the patient-related data; and a prediction module that input the learning data group into one or more machine learning models, respectively, to learn the machine learning models to predict mortality of the corresponding patient.

The mortality prediction device may further comprise a pretreatment module that excludes the patient-related data of the patient corresponding to exclusion conditions preset from the collected patient-related data.

The pretreatment module may determine whether the corresponding patient corresponds to the preset exclusion conditions based on one or more of time of death of a patient based on arrival at a hospital, whether the patient is treated after arrival at the hospital, whether the patient has trauma, whether the patient is irrecoverable, whether the patient is voluntarily discharged, the patient's diagnosis code, and whether the patient's identity is not confirmed.

The learning data generation module may divide patient-related data of patients who do not correspond to the preset exclusion conditions into patient-related data of deceased patients and patient-related data of survived patients, and extract a plurality of data preset from the patient-related data of deceased patients and patient-related data of survived patients, respectively, to generate a learning data group for one patient, and the prediction module may input the learning data group of deceased patients and the learning data group of survived patients into one or more machine learning models, respectively, to learn the machine learning models to predict mortality of the corresponding patient.

The learning data generation module may generate a learning data group for the corresponding decreased patient and a learning data group for the corresponding survived patient, by extracting the patient's age, emergency patient classification level, intentionality information, injury mechanism information, presence or absence of emergency symptoms, AVPU (Alert Verbal Pain Unresponsive) scale, gender, preset vital signs, and ICD-10 code, from the patient-related data of deceased patients and the patient-related data of survived patients.

The prediction module may input the learning data group for deceased patients and the learning data group for survived patients into a plurality of machine learning models, respectively, to perform performance evaluation for the plurality of machine learning models, and according to the performance results, select one or more machine learning models among the plurality of machine learning models.

The prediction module may calculate a first performance evaluation score of each machine learning model by calculating the accuracy, sensitivity, and specificity of each machine learning model based on the prediction results of the plurality of machine learning models, and adding them up, and
generate an ROC (Receiver Operating Characteristic) curve for the prediction result of each machine learning model based on the prediction result of each machine learning model, and calculate a second performance evaluation score of each machine learning model based on an AUC (Area Under the Curve) value from the ROC curve, and
select one or more machine learning models based on an overall evaluation score that is the sum of the first performance evaluation score and the second performance evaluation score.

The prediction module may calculate importance of each variable included in the learning data group, and weigh the corresponding variable depending on the importance of the variables included in the learning data group.

The mortality prediction method of trauma patients according to one example disclosed, is a method performed in a computing device equipped with one or more processors, and a memory storing one or more programs executed by the one or more processors, which comprises collecting patient-related data of patients visiting an emergency department for a certain period of time; generating a learning data group for one patient by extracting a plurality of data preset from the patient-related data; and inputting the learning data group into one or more machine learning models, respectively, to learn the machine learning models to predict mortality of the corresponding patient.

According to the disclosed examples, by predicting mortality of trauma patients visiting an emergency department, they can help make quick and accurate decisions to reduce the golden time of patient treatment.

### BRIEF DESCRIPTION OF THE DRAWINGS

FIG. 1 is a block diagram showing the configuration of the mortality prediction device of trauma patients according to one example of the present invention.

FIG. 2 and FIG. 3 are diagrams showing the learning data group of the patient based on the patient-related data of deceased patients and patient-related data of survived patients in one example of the present invention.

FIG. 4 is a diagram showing importance of variables included in the learning data group in one example of the present invention.

FIG. 5 is a flowchart showing the mortality prediction method of trauma patients according to one example of the present invention.

FIG. 6 is a block diagram for illustrating and describing a computing environment comprising a computing device suitable for being used in exemplary examples.

### DETAILED DESCRIPTION

Hereinafter, specific embodiments of the present invention will be described with reference to drawings. The following detailed description is provided to help a comprehensive understanding of the methods, devices, and/or systems described in the present description. However, these are only examples, and the present invention is not limited thereto.

In describing the examples of the present invention, when it is judged that a detailed description of the prior art related to the present invention may unnecessarily obscure the gist of the present invention, the detailed description will be omitted. In addition, the terms described below are terms defined in consideration of their functions in the present invention, and may vary depending on the intention of the user or operator or custom or the like. Therefore, the definitions should be made based on the contents throughout the present description. The terms used in the detailed description are only for describing the examples of the present invention, and should never be limited. Unless clearly used otherwise, expressions in the singular form include the meaning of the plural form. In the present description, expressions such as “comprising” or “having” are intended to indicate certain characteristics, numbers, steps, operations, elements, parts or combinations thereof, and it should not be interpreted to exclude the existence or possibility of one or more other characteristics, numbers, steps, operations, elements, parts or combinations thereof other than those described.

In addition, the terms such as first, second, and the like may be used to describe various components, but the components should not be limited by the terms. The terms may be used for the purpose of distinguishing one component from other components. For example, without departing from the scope of the present invention, the first component may be named the second component, and similarly, the second component may also be named the first component.

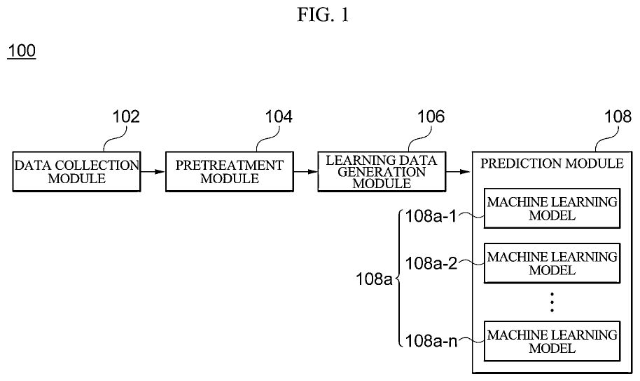

FIG. 1 is a block diagram showing the configuration of the mortality prediction device of trauma patients according to one example of the present invention.

Referring to FIG. 1, the mortality prediction device of trauma patients (100) may comprise a data collection module (102), a pretreatment module (104), a learning data generation module (106), and a prediction module (108). The mortality prediction device (100) may predict mortality of trauma patients based on artificial neural network technology.

The data collection module (102) may collect patient-related data of all patients visiting an emergency department for a certain period of time. Herein, the patient-related data may include all data of patients to be recorded at a hospital, when patients visit an emergency department.

The data collection module (102) may collect patient-related data of patients visiting an emergency department of each hospital by connecting with several hospitals. In one example, the data collection module (102) may collect patient-related data of each patient visiting an emergency department from NEDIS (National Emergency Department Information System).

The pretreatment module (104) may exclude patient-related data of patients corresponding to exclusion conditions preset from the collected patient-related data. The pretreatment module (104) may deliver patient-related data of the other patients excluding the patient-related data of the corresponding patient into the learning data generation module (106). In other words, the patient-related data which does not correspond to the preset exclusion conditions constitute a learning data set.

Specifically, the pretreatment module (104) may determine whether the corresponding patient corresponds to the preset exclusion conditions based on one or more of time of death of a patient based on arrival at a hospital, whether the patient is treated after arrival at the hospital, whether the patient has trauma, whether the patient is irrecoverable, whether the patient is voluntarily discharged, the patient's diagnosis code, and whether the patient's identity is not confirmed.

For example, the pretreatment module (104) may determine that the corresponding patient corresponds to the preset exclusion conditions, when a patient visiting an emergency department is dead before arrival to a hospital or dead at the arrival to a hospital. The pretreatment module (104) may determine that the corresponding patient corresponds to the preset exclusion conditions, when a patient arrives at a hospital alive but suffers cardiac arrest or dies without treatment such as cardiopulmonary resuscitation, and the like.

The pretreatment module (104) may determine that the corresponding patient corresponds to the preset exclusion conditions, when a patient visiting an emergency department has no trauma, or is an end stage disease patient such as cancer. The pretreatment module (104) may determine that the corresponding patient corresponds to the preset exclusion conditions, when a patient visiting an emergency department is a patient leaving a hospital as recovery is impossible or a patient discharging voluntarily.

The pretreatment module (104) may determine that the corresponding patient corresponds to the preset exclusion conditions, when it is difficult to confirm identity of a patient visiting an emergency department, or there is no trauma-related code in the patient's diagnosis code, or the patient's diagnosis code is a code related to frostbite, poisoning, unspecified injury or complications.

The learning data generation module (106) may constitute a learning data set to learn one or more machine learning models (108a) comprised in the prediction module (108) based on the patient-related data delivered from the pretreatment module (104) (i.e., patient-related data of a patient who does not correspond to the preset exclusion conditions).

The learning data generation module (106) may divide the patient-related data into cases in which the corresponding patient is dead and survived. In other words, the patient-related data may be divided into patient-related data of deceased patients and patient-related data of survived patients. The learning data generation module (106) may perform labeling for whether a patient is deceased or survived, respectively, on each patient-related data.

The learning data generation module (106) may generate a learning data group for one patient by extracting a plurality of data preset from the patient-related data of deceased patients and patient-related data of survived patients, respectively.

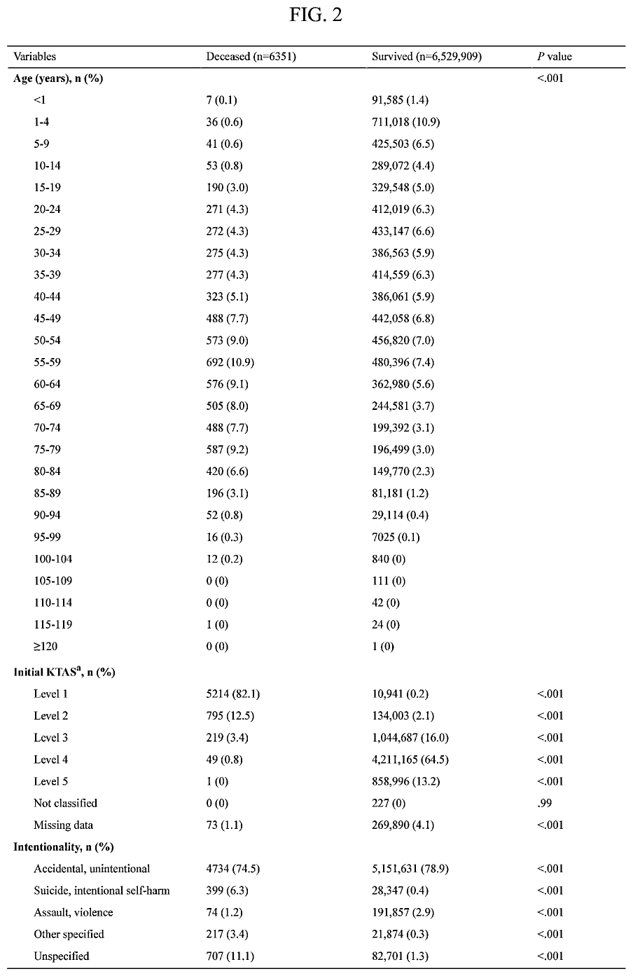

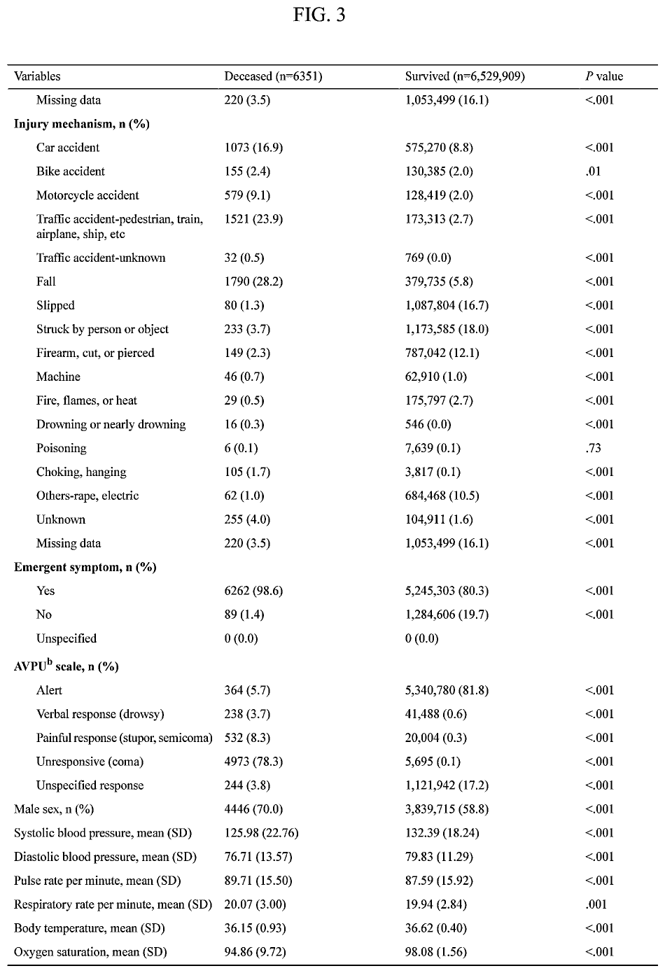

FIG. 2 and FIG. 3 are diagrams showing the learning data group of the patient based on the patient-related data of deceased patients and patient-related data of survived patients in one example of the present invention. Herein, a case in which cases of deceased patients were 6351 and cases of survived patients were 6,529,909 was shown.

Referring to FIG. 2 and FIG. 3, the learning data generation module (106) may extract the age of the corresponding patient from the patient-related data of deceased patients and the patient-related data of survived patients, respectively. In one example, the learning data generation module (106) may classify the age of the extracted patient into the preset age group (for example, age group in a 5-year unit).

The learning data generation module (106) may extract an emergency patient classification level of the corresponding patient from the patient-related data of deceased patients and the patient-related data of survived patients, respectively. For example, the learning data generation module (106) may extract KTAS (Korean Triage and Acuity Scale) classification level (Level 1˜Level 5) of each patient.

The learning data generation module (106) may extract intentionality information of the corresponding patient from the patient-related data of deceased patients and the patient-related data of survived patients, respectively. Herein, intentionality may include information about why the corresponding patient was injured. For example, intentionality may be classified into accidental, suicide, assault, and the like.

The learning data generation module (106) may extract injury mechanism information of the corresponding patient from the patient-related data of deceased patients and the patient-related data of survived patients, respectively. Herein, the injury mechanism may include specific information about by which accident the patient was injured. For example, the injury mechanism may be classified into car accident, bike accident, motorcycle accident, traffic accident, fall, slipped, struck by person or object, firearm/cut/pierced, machine, fire/flames/heat, poisoning, choking, and others-rape, and the like.

The learning data generation module (106) may extract whether there are emergency symptoms of the corresponding patient (yes or no) from the patient-related data of deceased patients and the patient-related data of survived patients, respectively.

The learning data generation module (106) may extract an AVPU (Alert Verbal Pain Unresponsive) scale of each corresponding patient from the patient-related data of deceased patients and the patient-related data of survived patients. In other words, the learning data generation module (106) may classify the corresponding patient into Alert patient (patient who is conscious), Verbal patient (patient who is semi-conscious and respond to whispers or voice stimuli), Pain patient (patient who respond only to painful stimuli), and Unresponsive patient (unconscious patient who do not respond to any type of stimuli) depending on the AVPU scale.

The learning data generation module (106) may extract the gender of each corresponding patient from the patient-related data of deceased patients and the patient-related data of survived patients.

The learning data generation module (106) may extract the preset vital signs of each corresponding patient from the patient-related data of deceased patients and the patient-related data of survived patients. In one example, the vital signs may include a systolic blood pressure, a diastolic blood pressure, a pulse rate per minute, a respiratory rate per minute, a body temperature, and an oxygen saturation, and the like.

In addition, the learning data generation module (106) may extract the ICD-10 code of each corresponding patient from the patient-related data of deceased patients and the patient-related data of survived patients. The learning data generation module (106) may extract information on to which category the corresponding patient belongs among about 865 categories of the ICD-10 code which starts with S or T.

The learning data generation module (106) may generate a learning data group for the corresponding deceased patient by extracting the patient's age, emergency patient classification level, intentionality information, injury mechanism information, presence or absence of emergency symptoms, AVPU (Alert Verbal Pain Unresponsive) scale, gender, preset vital signs, and ICD-10 code, from the patient-related data of deceased patients. The learning data generation module (106) may label the learning data group for the deceased patients as deceased patients. The ICD-10 code means the International Statistical Classification of Diseases and Health Problems code.

Furthermore, the learning data generation module (106) may generate a learning data group for the corresponding survived patient by extracting the patient's age, emergency patient classification level, intentionality information, injury mechanism information, presence or absence of emergency symptoms, AVPU (Alert Verbal Pain Unresponsive) scale, gender, preset vital signs, and ICD-10 code, from the patient-related data of deceased patients. The learning data generation module (106) may label the learning data group for the survived patients as survived patient.

On the other hand, the number of the patient-related data for deceased patients is significantly smaller than the patient-related data for survived patients, so the learning data generation module (106) may generate a learning data group after performing oversampling for the patient-related data for deceased patients.

In one example, the learning data generation module (106) may select any patient-related data from the distribution of the patient-related data for deceased patients, and based on the selected patient-related data, extract a certain number of nearest neighbors adjacent thereto, and generate random patient-related data so as to be arranged between the any patient-related data used as a standard and the extracted patient-related data of the neighbors. Then, the generated patient-related data may be randomly generated so as to have a uniform distribution value from the preset any patient-related data used as the preset standard and the extracted patient-related data of the neighbors. When such a process is repeatedly performed, the patient-related for deceased patients may be increased.

The prediction module (108) may learn one or more machine learning models (106a) to predict mortality of the patient based on the learning data group for deceased patients and the learning data group for survived patients which are generated in the learning data generation module (106).

In one example, the prediction module (108) may input the learning data group for deceased patients and the learning data group for survived patients into a plurality of machine learning models (106a-1, 106a-2, . . . , 106a-n), respectively, thereby allowing each machine learning model to predict mortality of the corresponding patient. In one example, the machine learning model may include AdaBoost (adaptive boosting), XGBoost (extreme gradient boosting), LightGBM (light gradient boosting), GBM (gradient boosting machine), ERT (extremely random trees), LR (logistic regression), RF (random forest), and DNN (deep neural network), and the like.

The prediction module (108) may perform the performance evaluation for the plurality of machine learning models, and select one or more machine learning models among the plurality of machine learning models depending on the performance evaluation results.

The prediction module (108) may calculate preset performance evaluation elements from the prediction results (mortality prediction results of patients) of the plurality of machine learning models. The prediction module (108) may calculate a first performance evaluation score of each machine learning model by calculating the accuracy, sensitivity, and specificity of each machine learning model based on the prediction results of the plurality of machine learning models, and adding them up.

Herein, the accuracy of the machine learning model is an index that indicates how accurately the corresponding machine learning model predicts. The accuracy of the machine learning model is a ratio of the number of correct cases to the total number of cases predicted by the machine learning model, and may be represented by the following Equation 1.

$$Accuracy = \frac{TP + TN}{TP + FP + TN + FN} \qquad ( Equation 1 )$$

TP (True Positive): a case in which the answer value is True, but the machine learning model determines the prediction as True
TN (True Negative): a case in which the answer value is False, but the machine learning model determines the prediction as False
FP (False Positive): a case in which the answer value is False, but the machine learning model determines the prediction as True
FN (False Negative): a case in which the answer value is True, but the machine learning model determines the prediction as False

In addition, the sensitivity of the machine learning model is a ratio of the number of correct cases among those that are predicted by the corresponding machine learning model for the positives, and may be represented by the following Equation 2.

$$Sensitivity = \frac{TP}{( TP - FN )} \qquad ( Equation 2 )$$

Moreover, the specificity oof the machine learning model is a ratio of the number of correct cases among those that are predicted by the corresponding machine learning model for the negatives, and may be represented by the following Equation 3.

$$Specificity = \frac{TN}{( TN + FP )} \qquad ( Equation 3 )$$

The prediction module (108) may generate an ROC (Receiver Operating Characteristic) curve for the prediction result of each machine learning model based on the prediction result of each machine learning model, and calculate a second performance evaluation score of each machine learning model based on an AUC (Area Under the Curve) value from the ROC curve. The AUC value may be a value indicating the total area under the ROC curve. The prediction module (108) may set the second performance evaluation score higher, as the AUC value is higher.

The prediction module (108) may select one or more machine learning models based on an overall evaluation score that is the sum of the first performance evaluation score and the second performance evaluation score, after calculating the first performance evaluation score and the second performance evaluation score, respectively, for each machine learning model. The prediction module (108) may set learning of the selected machine learning model as the model to predict mortality for trauma patients, and when the learning of the selected machine learning model is completed, in case that trauma patients enter an emergency department, the mortality of each trauma patient may be predicted.

On the other hand, the prediction module (108) may calculate importance of each variable (i.e., age, emergency patient classification level, intentionality, injury mechanism, emergency symptoms, AVPU scale, gender, preset vital signs, and ICD-10 code, etc.) in the learning data groups of deceased patients and survived patients during learning of the machine learning model. In other words, the prediction module (108) may calculate the degree of influence (i.e., importance) of each variable included in the learning data group on the prediction results of the machine learning model. For example, the prediction module (108) may calculate importance of a specific variable by inputting into the machine learning model after excluding the specific variable from the learning data group and confirming the prediction results of the machine learning model.

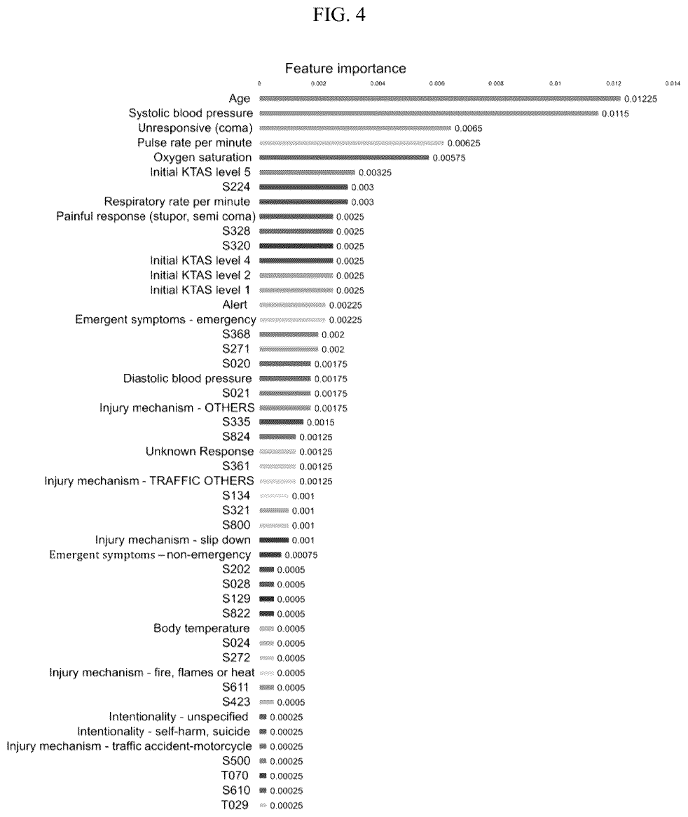

FIG. 4 is a diagram showing importance of variables included in the learning data group in one example of the present invention. Referring to FIG. 4, it can be confirmed that the patient's age is the most important variable, and then, systolic blood pressure, unresponsive, pulse rate per minute and oxygen saturation are important variables in order. The prediction module (108) may learn the machine learning model by weighting depending on the importance of the variables included in the learning data group.

According to the disclosed examples, by predicting mortality of trauma patients visiting an emergency department, they can help make quick and accurate decisions to reduce the golden time of patient treatment.

In the present description, the term module may mean a functional and structural combination of hardware to perform the technical idea of the present invention and software to operate the hardware. For example, the “module” may mean a logical unit of a predetermined code and a hardware resource for performing the predetermined code, and does not necessarily mean a physically connected code, or mean one type of hardware.

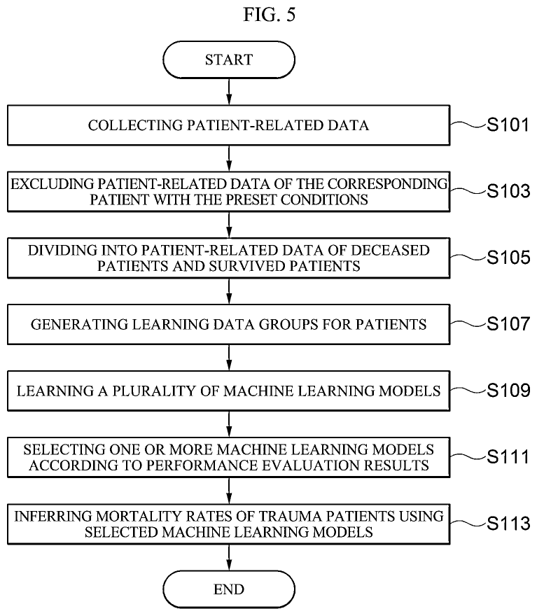

FIG. 5 is a flowchart showing the mortality prediction method of trauma patients according to one example of the present invention. In the flowchart shown, the method is described by dividing into a plurality of steps, but at least some of the steps may be performed in a different order, or performed together in combination with other steps, omitted, or performed by dividing into detailed steps, or performed by adding one or more steps that are not shown.

Referring to FIG. 5, the mortality prediction device (100) may collect patient-related data of patients visiting an emergency department for a certain period of time (S 101).

Next, the mortality prediction device (100) may exclude patent-related data of patients corresponding to preset exclusion conditions from the collected patient-related data (S 103).

Then, the mortality prediction device (100) may divide patient-related data into patient-related data of deceased patients and patient-related data of survived patients (S 105).

Next, the mortality prediction device (100) may generate a learning data group for one patient by extracting the preset plurality of data from the patient-related data of deceased patients and patient-related data of survived patients, respectively (S 107).

Then, the mortality prediction device (100) may input the learning data group of deceased patients and the learning data group of survived patients into a plurality of machine learning models to make the plurality of machine learning models to predict mortality of patients (S 109).

Then, the mortality prediction device (100) may perform the performance evaluation of the plurality of machine learning models based on the prediction results of the plurality of machine learning models, and select one or more machine learning models according to the results of the performance evaluation (S 111).

Next, the mortality prediction device (100) may infer mortality for trauma patients visiting an emergency department using the selected one or more machine learning models.

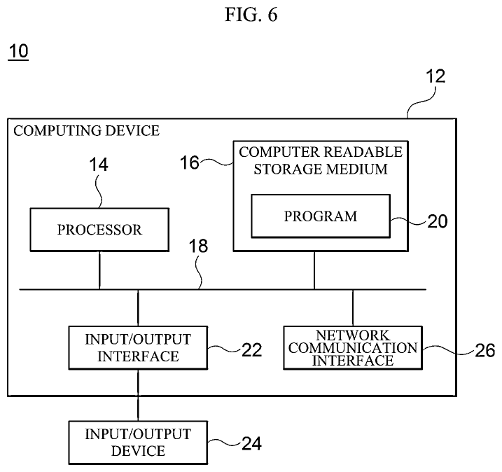

FIG. 6 is a block diagram for illustrating and describing a computing environment (10) comprising a computing device suitable for being used in exemplary examples. In the illustrated example, each component may have different functions and capabilities other than those described below, and may comprise additional components other than those described below.

The illustrated computing environment (10) comprises a computing device (12). In one example, the computing device (12) may be the mortality prediction device of trauma patients (100).

The computing device (12) comprises at least one processor (14), a computer readable storage medium (16) and a communication bus (18). The processor (14) may make the computing device (12) to operate according to the afore-mentioned exemplary examples. For example, the processor (14) may execute one or more programs stored in the computer readable storage medium (16). The one or more programs may comprise one or more computer executable instructions, and the computer executable instructions may be configured to make the computing device (12) to perform operations according to the exemplary examples, when executed by the processor (14).

The computer readable storage medium (16) is configured to store computer executable instructions or program codes, program data and/or other suitable forms of information. The programs (20) stored in the computer readable storage medium (16) comprise a set of instructions executable by the processor (14). In one example, the computer readable storage medium (16) may be a memory (volatile memory such as a random-access memory, non-volatile memory, or a suitable combination thereof), one or more magnetic disk storage devices, optical disk storage devices, flash memory devices, other forms of storage media that are accessed by the computing device (12) and store desired information, or a suitable combination thereof.

The communication bus (18) interconnects other various components of the computing device (12) by comprising the processor (14) and computer readable storage medium (16).

The computing device (12) may comprise one or more input/output interfaces (22) that provide interfaces for one or more input/output devices (24) and one or more network communication interfaces (26). The input/output interfaces (22) and network communication interfaces (26) are connected to the communication bus (18). The input/output devices (24) may be connected to other components of the computing device (12) through the input/output interfaces (22). The illustrative input/output devices (24) may comprise input devices such as pointing devices (mouse or trackpad, etc.), keyboards, touch input devices (touchpad or touchscreen, etc.), voice or sound input devices, various kinds of sensor devices and/or photographing devices, and/or output devices such as display devices, printers, speakers, and/or network cards. The illustrative input/output devices (24) may be comprised inside the computing device (12) as one component, and may be connected with the computing device (12) as a separate device distinguished from the computing device (12).

Representative examples of the present invention have been described in detail above, but those skilled in the art will understand that various modifications can be made to the afore-mentioned examples without departing from the scope of the present invention. Therefore, the scope of the present invention should not be limited to the described examples, and should be determined not only by the claims described below but also by equivalents of these claims.

What is claimed is:

1. A mortality prediction device of trauma patients equipped with one or more processors and a memory storing one or more programs executed by the one or more processors, the mortality prediction device comprising:
a data collection module configured to collect patient-related data of patients visiting an emergency department for a certain period of time;
a learning data generation module configured to generate a learning data group for one patient by extracting a plurality of data preset from the patient-related data; and
a prediction module configured to input the learning data group into one or more machine learning models, respectively, to learn the machine learning models to predict mortality of the corresponding patient.

2. The mortality prediction device of claim 1, wherein the mortality prediction device further comprises a pretreatment module that excludes the patient-related data of the patient corresponding to exclusion conditions preset from the collected patient-related data.

3. The mortality prediction device of claim 2, wherein the pretreatment module is configured to determines whether the corresponding patient corresponds to the preset exclusion conditions based on one or more of time of death of a patient based on arrival at a hospital, whether the patient is treated after arrival at the hospital, whether the patient has trauma, whether the patient is irrecoverable, whether the patient is voluntarily discharged, the patient's diagnosis code, and whether the patient's identity is not confirmed.

4. The mortality prediction device of claim 2, wherein the learning data generation module is configured to divide patient-related data of patients who do not correspond to the preset exclusion conditions into patient-related data of deceased patients and patient-related data of survived patients, and extracts a plurality of data preset from the patient-related data of deceased patients and patient-related data of survived patients, respectively, to generate a learning data group for one patient, and
the prediction module is configured to input the learning data group of deceased patients and the learning data group of survived patients into one or more machine learning models, respectively, to learn the machine learning models to predict mortality of the corresponding patient.

5. The mortality prediction device of claim 4, wherein the learning data generation module is configured to generate a learning data group for the corresponding decreased patient and a learning data group for the corresponding survived patient, by extracting the patient's age, emergency patient classification level, intentionality information, injury mechanism information, presence or absence of emergency symptoms, AVPU (Alert Verbal Pain Unresponsive) scale, gender, preset vital signs, and ICD-10 code, from the patient-related data of deceased patients and the patient-related data of survived patients.

6. The mortality prediction device of claim 5, wherein the prediction module is configured to input the learning data group for deceased patients and the learning data group for survived patients into a plurality of machine learning models, respectively, to perform performance evaluation for the plurality of machine learning models, and according to the performance results, selects one or more machine learning models among the plurality of machine learning models.

7. The mortality prediction device of trauma patients according to claim 6, wherein the prediction module is configured to:
calculate a first performance evaluation score of each machine learning model by calculating the accuracy, sensitivity, and specificity of each machine learning model based on the prediction results of the plurality of machine learning models, and adding them up;
generate an ROC (Receiver Operating Characteristic) curve for the prediction result of each machine learning model based on the prediction result of each machine learning model, and calculates a second performance evaluation score of each machine learning model based on an AUC (Area Under the Curve) value from the ROC curve; and
select one or more machine learning models based on an overall evaluation score that is the sum of the first performance evaluation score and the second performance evaluation score.

8. The mortality prediction device of trauma patients according to claim 5, wherein the prediction module is configured to calculate importance of each variable included in the learning data group, and weights the corresponding variable depending on the importance of the variables included in the learning data group.

9. A mortality prediction method of trauma patients, the method performed in a computing device equipped with one or more processors and a memory storing one or more programs executed by the one or more processors, the A mortality prediction method comprising:
collecting patient-related data of patients visiting an emergency department for a certain period of time;
generating a learning data group for one patient by extracting a plurality of data preset from the patient-related data; and
inputting the learning data group into one or more machine learning models, respectively, to learn the machine learning models to predict mortality of the corresponding patient.

10. The mortality prediction method of claim 9, further comprising pretreating that excludes the patient-related data of the patient corresponding to exclusion conditions preset from the collected patient-related data.

11. The mortality prediction method of claim 10, wherein the pretreating comprises determining whether the corresponding patient corresponds to the preset exclusion conditions based on one or more of time of death of a patient based on arrival at a hospital, whether the patient is treated after arrival at the hospital, whether the patient has trauma, whether the patient is irrecoverable, whether the patient is voluntarily discharged, the patient's diagnosis code, and whether the patient's identity is not confirmed.

12. The mortality prediction method of claim 10, wherein the generating of the learning data group comprises:
dividing patient-related data of patients who do not correspond to the preset exclusion conditions into patient-related data of deceased patients and patient-related data of survived patients; and
extracting a plurality of data preset from the patient-related data of deceased patients and patient-related data of survived patients, respectively, to generate a learning data group for one patient, and
the inputting of the learning data group comprises the learning data group of deceased patients and the learning data group of survived patients into one or more machine learning models, respectively, to learn the machine learning models to predict mortality of the corresponding patient.

13. The mortality prediction method of claim 12, wherein the generating of the learning data group comprises generating a learning data group for the corresponding decreased patient and a learning data group for the corresponding survived patient, by extracting the patient's age, emergency patient classification level, intentionality information, injury mechanism information, presence or absence of emergency symptoms, AVPU (Alert Verbal Pain Unresponsive) scale, gender, preset vital signs, and ICD-10 code, from the patient-related data of deceased patients and the patient-related data of survived patients.

14. The mortality prediction method of claim 13, wherein the learning further comprises:
inputting the learning data group for deceased patients and the learning data group for survived patients into a plurality of machine learning models, respectively, to perform performance evaluation for the plurality of machine learning models; and
selecting one or more machine learning models among the plurality of machine learning models, according to the performance results.

15. The mortality prediction method of claim 14, wherein the performing of the performance evaluation comprises:
calculating a first performance evaluation score of each machine learning model by calculating the accuracy, sensitivity, and specificity of each machine learning model based on the prediction results of the plurality of machine learning models, and adding them up; and
generating an ROC (Receiver Operating Characteristic) curve for the prediction result of each machine learning model based on the prediction result of each machine learning model, and calculates a second performance evaluation score of each machine learning model based on an AUC (Area Under the Curve) value from the ROC curve, and
the selecting comprises selecting one or more machine learning models based on an overall evaluation score that is the sum of the first performance evaluation score and the second performance evaluation score.

16. The mortality prediction method of claim 13, wherein the learning further comprises:
calculating importance of each variable included in the learning data group; and
weighing the corresponding variable depending on the importance of the variables included in the learning data group.

17. A computer program stored in a non-transitory computer readable storage medium, the computer program comprising one or more instructions executed by a computing device having one or more processors to perform:
collecting patient-related data of patients vising an emergency department for a certain period of time;
generating a learning data group for one patient by extracting a plurality of data preset from the patient-related data; and
inputting the learning data group into one or more machine learning models, respectively, to learn the machine learning models to predict mortality of the corresponding patient.
